(19) United States
(12) Patent Application Publication
(10) Pub. No.: US 2008/0193393 A1
(43) Pub. Date: Aug. 14, 2008

(54) Delivery System for Topically Applied Compounds

(75) Inventor: Nava Dayan, Fair Lawn, NJ (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: LIPO CHEMICALS INC., Peterson, NJ (US)

(21) Appl. No.: 11/662,996

(22) PCT Filed: Sep. 13, 2005
(86) PCT No.: PCT/US05/32749

§ 371 Date: Aug. 17, 2007

Related U.S. Application Data
(60) Provisional application No. 60/613,034, filed on Sep. 24, 2004.

(51) Int. Cl. A61K 8/18; A61K 47/44; A61K 47/12; A61K 31/56; A61K 36/28; A61Q 17/04; A61K 36/886; A61K 36/00; A61K 47/30

(52) U.S. Cl. 424/59; 514/783; 514/784; 514/772.3; 424/725; 514/169; 424/744; 424/764

(57) ABSTRACT

A delivery system for topically applied compounds is disclosed. The delivery system contains a fatty acid, a phospholipid, and an oil, and is activated by the addition of water. The delivery system is admixed with a topically applied compound and water to provide a composition suitable for application to the skin or hair. The relative amounts of delivery system ingredients provide round, flexible vesicles that allow penetration of the topically applied compound to the epidermis and dermis, vesicles having a partially ruptured membrane for a controlled delivery of the topically applied drug to the epidermis and dermis, completely ruptured vesicles in the form of lamellar sheets that allow the topically applied compound to be retained in the stratum corneum, and mixtures thereof.

### CROSS REFERENCE TO RELATED APPLICATIONS

This application claims the benefit of U.S. provisional patent application Ser. No. 60/613,034, filed Sep. 24, 2004.

### FIELD OF THE INVENTION

The present invention relates to delivery systems for the controlled release of a topically applied compound from a composition. More particularly, the present invention relates to a delivery system for the controlled release of a topically applied compound from a composition, wherein the composition comprises (a) a delivery system comprising a fatty acid, a phospholipid, and an oil, (b) a topically applied compound, and (c) water.

### BACKGROUND OF THE INVENTION

The administration of an active ingredient frequently is limited by natural barriers which prevent adequate introduction of the active ingredient to the desired target site because the barrier is not sufficiently permeable to the active ingredient. For topically applied compounds, the natural barrier is the skin.

Topically applied compounds fall into a variety of chemical classes and perform a variety of different functions. Several topically applied compounds are designed to perform their intended function on the surface of the skin. Other topically applied compounds are designed to penetrate the surface of the skin either to perform their intended function beneath the surface of the skin. However, the physical, protective barrier provided by the skin surface makes it difficult to effectively deliver topically applied compounds beneath the skin surface.

Mammalian skin comprises two distinct layers, i.e., the epidermis, of which the outermost layer is the stratum corneum composed of several layers of dead cells surrounded by lipophilic lamellar structures, and the dermis, which is vascular and below the living epidermis. The epidermis and dermis is where most vital functions of the skin are performed. Thus, for the topical delivery of a compound to the epidermis and dermis, the compound must pass through the stratum corneum.

A noninvasive administration of personal care products that perform their intended function beneath the skin surface would be advantageous in many cases. Therefore, substantial effort has been exerted to improve the skin permeability of various active agents. Topical administration of an active agent is easy, improves patient compliance, and may protect the compound from degradation. One method of increasing skin permeability is the use of chemical additives, such as solvents or surfactants.

The best known method for increasing penetration of an active agent through the skin is based on the use of penetration enhancers. Such penetration enhancers comprise nonionic materials (e.g., long-chain alcohols, surfactants, zwitter-ionic phospholipids), anionic materials (e.g., fatty acids), cationic long-chain amines, sulfoxides, as well as various amino derivatives, and amphoteric glycinates and betaines. Nevertheless, the problem of active agent penetration into the skin has not yet been solved satisfactorily.

Patent publications disclosing compositions or delivery systems that deliver a topically applied compound to a skin surface and/or that penetrate the skin surface include U.S. Patent Publication No. US2002/0048596; U.S. Pat. No. 6,165,520; U.S. Patent Publication No. US2003/0099694; and U.S. Patent Publication No. US2002/0064524.

The present application is directed to a delivery system for topically applied compounds, and to compositions containing the delivery system and a topically applied compound. The present delivery system allows an effective and facile application of the topically applied composition to the surface of the skin and depending on the composition allows retention of the topically applied compound either in the stratum corneum or in the epidermis and dermis by penetrating the skin surface.

### SUMMARY OF THE INVENTION

The present invention is directed to a delivery system for topically applied compounds, and to compositions comprising the delivery system and a topically applied compound. More particularly, the present invention is directed to a delivery system that allows application of a topically applied compound to the surface of the skin to penetrate either to the stratum corneum, or to the epidermis and dermis by penetrating the skin surface.

In accordance with the present invention, the delivery system is admixed with a topically applied compound and water to provide a composition suitable for application to the skin, including the scalp. This mixture then can be incorporated into a formulation, or formulation ingredients can be added thereto, for application and delivery of the topically applied compound.

In one aspect of the present invention, the relative amounts and identity of ingredients in the delivery system provide (a) round, flexible vesicles that allow penetration of the topically applied compound to the living epidermis and dermis, (b) vesicles having a partially ruptured membrane for the controlled delivery of the topically applied drug to the epidermis and dermis, (c) vesicles having a completely ruptured membrane creating lamellar sheets for a controlled delivery and retention of the topically applied compound to the stratum corneum, and (d) mixtures containing at least two of (a), (b), and (c).

Another aspect of the present invention is to provide a delivery system for topically applied compounds comprising a fatty acid, a phospholipid, and an oil. The delivery system typically is free of intentionally added surfactants, but can contain up to 2%, by weight, of a surfactant. More particularly, the delivery system comprises about 2% to about 50%, by weight, of a fatty acid; about 5% to about 50%, by weight, of a phospholipid; and about 20% to about 90%, by weight, of an oil.

Still another aspect of the present invention is to provide a composition comprising a delivery system of the present invention, a topically applied compound, and water, wherein (a) the topically applied compound is present in a sufficient amount to perform its intended function, and (b) the delivery system and water are present, respectively, in a weight ratio of about 1 to 1 to about 1 to 100.

Yet another aspect of the present invention is to provide a composition containing a present delivery system and a topically applied compound selected from the group consisting of a skin care compound, a topical drug, an antioxidant, a dye, a skin lightening compound, a self-tanning compound, an optical brightener, a deodorant, a fragrance, a sunscreen, an insect repellant, a drug, similar topically applied compounds, and mixtures thereof.

These and other aspects and novel features of the present invention will become apparent from the following detailed description of the preferred embodiments.

### BRIEF DESCRIPTION OF THE FIGURES

FIG. 1 is a transmission electron microscopy photograph showing round vesicles of the present invention;

FIG. 2 is a transmission electron microscopy photograph showing partially ruptured vesicles of the present invention; and

FIG. 3 is a transmission electron microscopy photograph showing completely ruptured vesicles creating lamellar sheets of the present invention; and

FIGS. 4-7 are transmission electron microscopy photograph showing the effect of oleic acid on the structural differences of the vesicles.

### DETAILED DESCRIPTION OF THE PREFERRED EMBODIMENTS

The present invention is directed to a delivery system for topically applied compounds, and to compositions comprising a present delivery system and a topically applied compound. In particular, the delivery system allows for the sustained release of a topically applied compound in the stratum corneum, and for the controlled release of a topically applied compound beneath the skin surface, e.g., in the epidermis and dermis.

As used herein, the term “delivery system” refers to a mixture of the fatty acid, phospholipid, and oil. The term “activated delivery system” refers to a delivery system diluted with water.

As used herein, the term “sustained release” means a release of a topically applied compound over an extended period, i.e., providing a continuous supply of topically applied compound at the desired target site. The term “controlled release” means release of a topically applied compound at a desired target site, i.e., stratum corneum or epidermis and dermis, due to a reservoir of the topically applied compound in the desired target site in subtissues.

A delivery system of the present invention comprises: (a) a fatty acid, (b) a phospholipid, and (c) an oil. The delivery system is activated by the addition of water. The delivery system is admixed with a topically active compound and water, and other optional ingredients, to provide a composition of the present invention. Depending upon the relative amounts of (a), (b), and (c), and the HLB (hydrophilic-lipophilic balance) of (b), the delivery system is present in a composition of the invention as (i) round vesicles, (ii) vesicles with a partially ruptured membrane, (iii) completely ruptured vesicles creating lamellar sheets that mimic the lamellar structure existing between the stratum corneum cells, and (iv) mixtures thereof.

The relative amounts of round vesicles, partially ruptured vesicles, and lamellar sheets allow for the controlled delivery of a topically applied drug to a desired target site, i.e., the skin surface or epidermis and dermis. A controlled delivery of the topically applied compound to the skin surface or epidermis and dermis can provide a sustained release of the topically applied compound over time.

1. Fatty Acid

A delivery system of the present invention comprises about 2% to about 50%, by weight, and preferably about 5% to about 40%, by weight, of a fatty acid. To achieve the full advantage of the present invention, the delivery system comprises about 10% to about 30%, by weight, of a fatty acid.

A fatty acid incorporated into a present delivery system can be a C₈ to C₂₆ fatty acid. The fatty acid can be saturated or can contain one or more carbon-carbon double bonds. Examples of fatty acids useful in the delivery system include, but are not limited to, capric acid, caprylic acid, decanoic acid, lauric acid, behenic acid, tallow acid, caproic acid, myristic acid, oleic acid, linoleic acid, stearic acid, isostearic acid, tall oil acid, coconut acid, pelargonic acid, linolenic acid, ricinoleic acid, palmitic acid, hydroxystearic acid, linseed acid, undecylenic acid, soy acid, and mixtures thereof. Additional fatty acids listed in the CTFA Cosmetic Ingredient Handbook, First Ed., J. Nikotakis, ed., The Cosmetic Toiletry, and Fragrance Association (1988), hereafter, the CTFA Handbook, pages 27 and 28, incorporated herein by reference.

2. Phospholipid

In addition to the fatty acid, a present delivery system comprises a phospholipid. The delivery system comprises about 5% to about 50%, by weight, and preferably, about 5% to about 35%, by weight, of a phospholipid. To achieve the full advantage of the present invention, the delivery system comprises about 10% to about 30%, by weight, of a phospholipid.

Phospholipids useful in the present invention are not limited. The delivery system, therefore, can be prepared using phosphatidylethanolamine (i.e., cephalin), phosphatidylcholine (i.e., lecithin), phosphatidylserine, phosphatidylinositol, phostidylglycerol, 3′-O-lysylphosphatidylglycerol, cardiolipin, sphingomyelin, and mixtures thereof, for example. In general, the phospholipid can be any glyceride esterified by C₆-C₂₄ fatty acids at the 1,2-positions and having a phosphoric acid ester residue at the 3 position.

Other useful phospholipids have a phosphoric acid ester residue containing a positive charge, typically a quaternary ammonium nitrogen. Such phospholipids include, but are not limited to, phosphatidylethanolamine, phosphatidylcholine, phosphatidylserine, and 3′-O-lysylphosphatidylglycerol.

It is not necessary to use a purified phospholipid. Commercial phospholipids, like commercial lecithin, can be used in the present invention, and, therefore, provide economies.

Phospholipids having different HLB values are available, and the HLB value of the phospholipid has an effect on the form of the vesicles present in a water-activated delivery system of the present invention. Phospholipids having a lower HLB, e.g., about 7, stabilize round vesicles, whereas phospholipids having a higher HLB stabilizes ruptured vesicles (e.g., HLB about 9) and lamellar sheets (e.g., HLB about 10). The HLB value of a compound is a well-known property to persons skilled in the art. HLB values of compounds are published and also can be calculated or determined experimentally. For example, see W. C. Griffin, J. Soc. Cosmetic Chem., 5, 294 (1954).

3. Oil

In addition to the fatty acid and phospholipid, a present delivery system comprises an oil. The oil can be a natural oil, a synthetic oil, or mixtures thereof. The delivery system comprises about 20% to about 90%, by weight, and preferably about 30% to about 85%, by weight, of an oil. To achieve the full advantage of the present invention, the delivery system comprises about 40% to about 80%, by weight, of an oil.

The identity of the oil is not limited. Examples of useful oils include, but are not limited to, rice bran oil, lanolin oil, linseed oil, coconut oil, olive oil, menhaden oil, castor oil, soybean oil, tall oil, rapeseed oil, palm oil, neatsfoot oil, eucalyptus oil, peppermint oil, rose oil, clove oil, lemon oil, pine oil, orange oil, almond oil, apricot kernel oil, avocado oil, chaulmoogra oil, cherry pit oil, cocoa butter, cod liver oil, corn oil, cottonseed oil, egg oil, ethiodized oil, grape seed oil, hazel nut oil, hybrid safflower oil, hydrogenated castor oil, hydrogenated coconut oil, hydrogenated cottonseed oil, hydrogenated menhaden oil, hydrogenated palm kernel oil, hydrogenated palm oil, hydrogenated peanut oil, hydrogenated shark liver oil, hydrogenated soybean oil, hydrogenated vegetable oil, jojoba oil, mink oil, moringa oil, olive husk oil, palm kernel oil, palm oil, peach kernel oil, peanut oil, pengawar djambi oil, safflower oil, sesame oil, shark liver oil, shea butter, sunflower seed oil, sweet almond oil, vegetable oil, walnut oil, wheat bran lipids, wheat germ oil, and mixtures thereof. Additional oils are listed in the CFTA Handbook, pages 23, 26, and 27, incorporated herein by reference.

The oil also can be a synthetic oil, like a hydrocarbon, e.g., mineral oil, 1-decene dimer, a polydecene, paraffin, petrolatum, or an isoparaffin, for example. Another class of synthetic oils is the silicone oils, like dimethicone, and the functional silicone oils, like dimethicone copolyol. The silicone oils have a viscosity of about 10 centipoise (cps) to about 600,000 cps, and typically about 350 cps to about 10,000 cps, at 25° C. Examples of silicone oils include dimethicone, dimethicone co-polyol, dimethiconol, simethicone, phenyl trimethicone, stearoxy dimethicone, trimethylsilylamodimethicone, an alkyl dimethicone copolyol, and a dimethicone having polyoxyethylene and/or polyoxypropylene side chains.

A delivery system of the present invention can contain an intentionally added surfactant, including anionic, cationic, nonionic, ampholytic, and amphoteric surfactants. The term “surfactant” herein means a surface active agent other than a phospholipid. In particular, “surfactants” means cleansing or detergent-type surfactants, including anionic, nonionic, cationic, ampholytic, and amphoteric surfactants. Typically, a present delivery system is essentially free of a surfactant, but can contain 0% to about 2%, by weight, of a surfactant.

A present delivery system also can include optional ingredients. For example, a delivery system can include an antioxidant, such as vitamin E and/or a vitamin E derivative. Other optional ingredients include, but are not limited to, fragrances, preservatives, dyes, and cholesterol esters. An optional ingredient is present in a sufficient amount to perform its intended function, and typically is present in an amount of 0% to about 1%, by weight, of the delivery system. In total, optional ingredients are present in a delivery system in an amount of 0% to about 10%, by weight, of the delivery system.

Typically, the weight ratio of fatty acid to phospholipid in the delivery system is about 0.5 to 1 to about 2 to 1. However, the weight ratio of fatty acid to phospholipid can be about 0.2 to 1 to about 5 to 1. In preferred embodiments, the weight ratio of fatty acid to phospholipid in the delivery system is about 0.8 to 1 to about 1.2 to 1.

A delivery system of the present invention is prepared by simply admixing the fatty acid, phospholipid, and oil until a uniform composition is achieved. The following illustrate two delivery systems of the present invention.

| | |
|---|---|
| Ingredient | Wt % |

Example 1

| | |
|---|---|
| Oleic Acid | 10 |
| Lecithin | 10 |
| Rice Bran Oil | 79.5 |
| Vitamin E Acetate | 0.5 |

Example 2

| | |
|---|---|
| Oleic Acid | 20 |
| Linoleic Acid | 10 |
| Lecithin | 30 |
| Rice Bran Oil | 39.5 |
| Vitamin E Acetate | 0.5 |

The following Examples 3-5 were prepared, then activated by admixture with an equal volume of deionized water. Phospholipids having a different HLB value were used in each example. In Examples 3 and 5, a blend of lecithins was used to provide the desired HLB. Example 3 incorporated a phospholipid blend having an HLB of 7 and provided round vesicles after activation with water. Example 4, after activation, incorporated a lecithin having an HLB of 9 and provided ruptured vesicles. Example 5 utilized a lecithin blend having an HLB of 10.5, and provided lamellar sheets after activation with water.

| Ingredient | Example 3 | Example 4 | Example 5 |
|---|---|---|---|
| Oleic acid | 10¹⁾ | 20 | 30 |
| Lecithin 1 | 21.50 | — | 21.50 |
| Lecithin 2 | — | 43 | 21.50 |
| Lecithin 3 | 21.50 | — | — |
| Jojoba oil | 47 | 37 | 27 |
| Preservative | 0.5 | 0.5 | 0.5 |
| LIPOSORB ™ L-20 | 1.0 | 1.0 | 1.0 |
| (Polysorbate 20) | | | |

¹⁾weights of ingredients in grams.

The delivery system of the present invention is useful in personal care, cosmetic, and pharmaceutical industries. The present delivery systems provide a controlled release and targeted delivery of topically applied compounds, such as fragrances, pigments, skin treatment agents, topical drugs, and similar topically applied compounds.

A delivery system of the present invention is admixed with water at room temperature or with heating to form an activated delivery system. Typically, a delivery system of the present invention is admixed with water or other vehicle containing water and a topically applied compound to provide a composition of the present invention. Water is added to the delivery system to provide a weight ratio of delivery system to water of about 1 to 1 to about 1 to 100, and preferably about 1 to about 50. More preferably, the weight ratio of delivery system to water is about 1 to 1 to about 1 to 10. This mixture then can be added to additional water and formulation ingredients, or water and additional ingredients can be added to the mixture to provide a final composition for application to the skin.

The addition of water to the delivery system activates the delivery system, and enables the formation of different structures that deliver the topically applied compound. As discussed hereafter, the relative amounts of fatty acid, phospholipid, and oil, and the HLB of the phospholipid, correlates to the predominant form of vesicles in the emulsion, i.e., round vesicles, partially ruptured vesicles, and completely ruptured vesicles as lamellar sheets. Typically, each of these forms is present in any activated delivery system. Changing the relative amounts or identity of fatty acid, phospholipid, and oil in the delivery system alters the distribution of the round, partially ruptured, and lamellar sheets, which in turn alters the ability of the composition to penetrate the skin surface to the epidermis and dermis. Overall, a controlled delivery of the topically applied compound to a desired target site can be achieved.

In addition to adding water to the delivery system, a topically applied compound typically is added to the delivery system. Conventionally, the topically active compound is added to the delivery system prior to the addition of water. Thus, the vesicles formed by the addition of water encapsulate or otherwise incorporate the topically applied compound for a controlled and/or sustained release of the topically applied compound.

A topically applied compound is incorporated in a composition of the present invention in a sufficient amount to perform its intended function. The specific amount of topically applied compound in a composition can vary widely, from a very small amount of a therapeutic drug to a relatively high amount of a skin care compound, e.g., an antiperspirant. The amount of topically applied compound included in a present composition is well known to persons skilled in the art based on the identity of the compound and its intended use.

In accordance with an important feature of the present invention, the topically applied compound can be any of a wide variety of compounds, either water soluble or oil soluble.

The topically applied compound, therefore, can be one of, or a mixture of, a cosmetic compound, a medicinally active compound, a compound used in cosmetics, personal care, or any other compound that is useful upon topical application to the skin. Such topically active agents include, but are not limited to, deodorants, skin-care compounds, plant extracts, antioxidants, insect repellents, counter-irritants, vitamins, steroids, retinoids, antibacterial compounds, antifungal compounds, antiinflammatory compounds, antibiotics, topical anesthetics, sunscreens, optical brighteners, and other cosmetic and medicinal topically effective compounds.

For example, a skin conditioner can be the topically applied compound. Skin conditioning agents include, but are not limited to, humectants, such a fructose, glucose, glycerin, propylene glycol, glycereth-26, mannitol, urea, pyrrolidone carboxylic acid, hydrolyzed lecithin, coco-betaine, cysteine hydrochloride, glucamine, PPG-15, sodium gluconate, potassium aspartate, oleyl betaine, thiamine hydrochloride, sodium hyaluronate, hydrolyzed proteins, hydrolyzed keratin, amino acids, amine oxides, water-soluble derivatives of vitamins A, E, and D, amino-functional silicones, ethoxylated glycerin, alpha-hydroxy acids and salts thereof, fatty oil derivatives, such as PEG-24 hydrogenated lanolin, and mixtures thereof. Numerous other skin conditioners are listed in the CTFA Cosmetic Ingredient Handbook, First Ed., J. Nikotakis, ed., The Cosmetic, Toiletry and Fragrance Association (1988), (hereafter CTFA Handbook), pages 79-84, incorporated herein by reference.

The skin conditioner also can be a water-insoluble ester having at least 10 carbon atoms, and preferably 10 to about 32 carbon atoms. Suitable esters include those comprising an aliphatic alcohol having about eight to about twenty carbon atoms and an aliphatic or aromatic carboxylic acid including from two to about twelve carbon atoms, or conversely, an aliphatic alcohol having two to about twelve carbon atoms with an aliphatic or aromatic carboxylic acid including about eight to about twenty carbon atoms. The ester is either straight-chained or branched. Suitable esters, therefore, include, for example, but are not limited to:

(a) aliphatic monohydric alcohol esters, including, but not limited to:

myristyl propionate,

isopropyl isostearate,

isopropyl myristate,

isopropyl palmitate,

cetyl acetate,

cetyl propionate,

cetyl stearate,

isodecyl neopentanoate,

cetyl octanoate,

isocetyl stearate;

(b) aliphatic di- and tri-esters of poly-carboxylic acid, including, but not limited to:

diisopropyl adipate,

diisostearyl fumarate,

dioctyl adipate, and

triisostearyl citrate;

(c) aliphatic polyhydric alcohol esters, including, but not limited to:

propylene glycol dipelargonate;

(d) aliphatic esters of aromatic acids, including, but not limited to:

C₁₂-C₁₅ alcohol esters of benzoic acid,

octyl salicylate,

sucrose benzoate, and

dioctyl phthalate.

Numerous other esters are listed in the CTFA Handbook, at pages 24 through 26, incorporated herein by reference and are available from Lipo Chemicals Inc. as LIPONATE™, LIPO polyglycol, and LIPOVOL™ products.

The topically applied compound also can be an antioxidant, like ascorbic acid or erythorbic acid, or an optical brightener. In addition, a self-tanning compound, like dihydroxyacetone, can be the topically applied agent.

Optical brighteners useful as the topically applied compound can be any compound capable of absorbing an invisible UV portion of the daylight spectrum, and converting this energy into the longer visible wavelength portion of the spectrum. The optical brightener is colorless on the substrate, and does not absorb energy in the visible part of the spectrum. The optical brightener typically is a derivative of stilbene or 4,4′-diaminostilbene, biphenyl, a 5-membered heterocycle, e.g., triazole, oxazole, or imidazole, or a 6-membered heterocycle, e.g., a coumarin, a naphthalamide, or an s-triazine. The optical brighteners are available under a variety of tradenames, such as TINOPAL®, LEUCOPHOR®, and CALCOFLUOR®. Specific fluorescent compounds include, but are not limited to, TINOPAL® 5BM, CALCOFLUOR® CG, and LEUCOPHOR® BSB.

The topically applied compound also can be a deodorant or antiperspirant compound, such as an astringent salt or a bioactive compound. The astringent salts include organic and inorganic salts of aluminum, zirconium, zinc, and mixtures thereof. The anion of the astringent salt can be, for example, sulfate, chloride, chlorohydroxide, formate, lactate, benzyl sulfonate, or phenyl sulfonate. Exemplary classes of antiperspirant astringent salts include aluminum halides, aluminum hydroxyhalides, zirconyl oxyhalides, zirconyl hydroxyhalides, and mixtures thereof.

Exemplary aluminum salts include aluminum chloride and the aluminum hydroxyhalides having the general formula Al₂(OH)ₓQ_{y}.XH₂O, wherein Q is chlorine, bromine, or iodine; x is about 2 to about 5; x+y is about 6, wherein x and y are not necessarily integers; and X is about 1 to about 6. Exemplary zirconium compounds include zirconium oxy salts and zirconium hydroxy salts also referred to as zirconyl salts and zirconyl hydroxy salts, and represented by the general empirical formula ZrO(OH)_{2-nz}L_{z}, wherein z varies from about 0.9 to about 2 and is not necessarily an integer; n is the valence of L; 2-nz is greater than or equal to 0; and L is selected from the group consisting of halides, nitrate, sulfamate, sulfate, and mixtures thereof.

Exemplary deodorant compounds, therefore, include, but are not limited to, aluminum bromohydrate, potassium alum, sodium aluminum chlorohydroxy lactate, aluminum sulfate, aluminum chlorohydrate, aluminum-zirconium tetrachlorohydrate, an aluminum-zirconium polychlorohydrate complexed with glycine, aluminum-zirconium trichlorohydrate, aluminum-zirconium octachlorohydrate, aluminum sesquichlorohydrate, aluminum sesquichlorohydrex PG, aluminum chlorohydrex PEG, aluminum zirconium octachlorohydrex glycine complex, aluminum zirconium pentachlorohydrex glycine complex, aluminum zirconium tetrachlorohydrex glycine complex, aluminum zirconium trichlorohydrex glycine complex, aluminum chlorohydrex PG, zirconium chlorohydrate, aluminum dichlorohydrate, aluminum dichloxohydrex PEG, aluminum dichlorohydrex PG, aluminum sesquichlorohydrex PG, aluminum chloride, aluminum zirconium pentachlorohydrate, chlorophyllin copper complex, numerous other useful antiperspirant compounds listed in the CTFA Handbook at page 56, incorporated herein by reference, and mixtures thereof. The active agent also can be a fragrance that acts as a deodorizer by masking malodors. Numerous fragrance compounds are listed in the CTFA Handbook, pages 69-70, incorporated herein by reference.

In addition, other compounds can be included as the topically applied compound in an amount sufficient to perform their intended function. For example, if the composition is intended to be a sunscreen, then compounds such as benzophenone-3, trihydroxycinnamic acid and salts, tannic acid, uric acids, quinine salts, dihydroxy naphtholic acid, an anthranilate, diethanolamine methoxy-cinnamate, p-aminobenzoic acid, phenylbenzimidazole sulfonic acid, PEG-25, p-aminobenzoic acid, or tri-ethanolamine salicylate can be used as the topically applied compound.

Further, sunscreen compounds such as di-oxybenzone, ethyl 4-[bis(hydroxypropyl)]aminobenzoate, glyceryl aminobenzoate, homosalate, methyl anthranilate, octocrylene, octyl methoxycinnamate, octyl salicylate, oxybenzone, padimate O, red petro-latum, titanium dioxide, 4-menthylbenzylidene camphor, benzophenone-1, benzophenone-2, benzophenone-6, benzophenone-12, isopropyl dibenzoyl methane, butyl methoxydibenzoylmethane, zotocrylene, or zinc oxide can be used as the topically applied compound. Other sunscreen compounds are listed in CTFA Handbook, pages 86 and 87, incorporated herein by reference.

Similarly, topically applied drugs, like antifungal compounds, antibacterial compounds, anti-inflammatory compounds, topical anesthetics, skin rash, skin disease, and dermatitis medications, and antiitch and irritation-reducing compounds can be used as the active agent in the compositions of the present invention. For example, analgesics such as benzocaine, dyclonine hydrochloride, aloe vera, and the like; anesthetics such as butamben picrate, lidocaine hydrochloride, xylocaine, and the like; antibacterials and antiseptics, such as povidone-iodine, polymyxin b sulfate-bacitracin, zinc-neomycin sulfate-hydrocortisone, chloramphenicol, ethyl-benzethonium chloride, erythromycin, and the like; antiparasitics, such as lindane; essentially all dermatologicals, like acne preparations, such as benzoyl peroxide, erythromycin benzoyl peroxide, clindamycin phosphate, 5,7-dichloro-8-hydroxyquinoline, and the like; antiinflammatory agents, such as alclometasone dipropionate, betamethasone valerate, and the like; burn relief ointments, such as o-amino-p-toluenesulfonamide monoacetate, and the like; depigmenting agents, such as monobenzone; dermatitis relief agents, such as the active steroid amcinonide, diflorasone diacetate, hydrocortisone, and the like; diaper rash relief agents, such as methylbenzethonium chloride, and the like; emollients and moisturizers, such as mineral oil, PEG-4 dilaurate, lanolin oil, petrolatum, mineral wax, and the like; fungicides, such as butocouazole nitrate, haloprogin, clotrimazole, and the like; herpes treatment drugs, such as O-[(2-hydroxymethyl)-methyl]guanine; pruritic medications, such as alclometasone dipropionate, betamethasone valerate, isopropyl myristate MSD, and the like; psoriasis, seborrhea, and scabicide agents, such as anthralin, methoxsalen, coal tar, and the like; steroids, such as 2-(acetyloxy)-9-fluoro-1′,2′,3′,4′-tetrahydro-11-hydroxypregna-1,4-dieno-[16,17-b]naphthalene-3,20-dione and 21-chloro-9-fluoro-1′,2′,3′,4′-tetrahydro-11b-hydroxypregna-1,4-dieno-[16,17-b]naphthalene-3,20-dione. Any other medication capable of topical administration, like skin bleaching agents, skin protectants, such as allantoin, and antiacne agents, such as salicylic acid, also can be incorporated in a composition of the present invention in an amount sufficient to perform its intended function. Other topically applied compounds are listed in Remington's Pharmaceutical Sciences, 17th Ed., Mack Publishing Co., Easton, Pa. (1985), pages 773-791 and pages 1054-1058 (hereinafter Remington's), incorporated herein by reference.

The topically applied compound also can be a plant extract. Nonlimiting plant extracts are those obtained from alfalfa, aloe vera, amla fruit, angelica root, anise seed, apple, apricot, artichoke leaf, asparagus root, banana, barberry, barley sprout, bee pollen, beet leaf, bilberry fruit, birch leaf, bitter melon, black currant leaf, black pepper, black walnut, blueberry, blackberry, burdock, carrot, cayenne, celery seed, cherry, chickwood, cola nut, corn silk, cranberry, dandelion root, elderberry, eucalyptus leaf, flax oil powder, ginger root, gingko leaf, ginseng, goldenrod, goldenseal, grape, grapefruit, guava, hibiscus, juniper, kiwi, kudzu, lemon, licorice root, lime, malt, marigold, myrrh, olive leaf, orange fruit, orange peel, oregano, papaya fruit, papaya leaf, passion fruit, peach, pear, pine bark, plum, pomegranate, prune, raspberry, rhubarb root, rosemary leaf, sage leaf, spearmint leaf, St. John's wart, strawberry, sweet cloves, tangerine, violet herb, watercress, watermelon, willow bark, wintergreen leaf, witch hazel bark, yohimbe, and yucca root.

The concentration of the delivery system relative to the entire composition is sufficient for the topically applied compound to perform its intended function, and typically is about 0.01% to about 50%, by weight, of the formulation. The amount of delivery system included in the composition is related to the identity of the topically applied compound and the amount of topically applied compound in the composition.

A composition of the present invention is prepared by admixing a present delivery system with a topically applied compound and water. The present compositions can include other ingredients traditionally included in cosmetic, medicinal, and other such compositions. These ingredients include, but are not limited to, dyes, fragrances, preservatives, surfactants, antioxidants, detackifying agents, and similar types of compounds. The ingredients are included in the composition in an amount sufficient to perform their intended function.

The following additional ingredients typically are included in a present composition, in combination with a present delivery system and topically applied compound. Each of these ingredients, and any other ingredient, is present in a sufficient amount to perform its intended function, without adversely affecting the composition or its efficacy.

For example, a present composition can contain a surfactant. The surfactant can be an anionic surfactant, a cationic surfactant, a nonionic surfactant, or a compatible mixture of surfactants. The surfactant also can be an ampholytic or amphoteric surfactant, which have anionic or cationic properties depending upon the pH of the composition.

A present composition also can contain a hydrotrope. A hydrotrope is a compound that has an ability to enhance the water solubility of other compounds. Specific examples of hydrotropes include, but are not limited to, sodium cumene sulfonate, ammonium cumene sulfonate, ammonium xylene sulfonate, potassium toluene sulfonate, sodium toluene sulfonate, sodium xylene sulfonate, toluene sulfonic acid, and xylene sulfonic acid. Other useful hydrotropes include sodium polynaphthalene sulfonate, sodium polystyrene sulfonate, sodium methyl naphthalene sulfonate, sodium camphor sulfonate, and disodium succinate.

A present composition further can contain a solvent. The solvent often is a water-soluble organic compound containing one to six, and typically one to three, hydroxyl groups, e.g., alcohols, diols, triols, and polyols. Specific examples of solvents include, but are not limited to, methanol, ethanol, isopropyl alcohol, n-butanol, n-propyl alcohol, ethylene glycol, propylene glycol, glycerol, diethylene glycol, dipropylene glycol, tripropylene glycol, hexylene glycol, butylene glycol, 1,2,6-hexanetriol, sorbitol, PEG-4, 1,5-pentanediol, similar hydroxyl-containing compounds, and mixtures thereof.

The solvent also can be an aprotic solvent, e.g., dimethyl sulfoxide or tetrahydrofuran, or a hydrocarbon solvent, e.g., an aliphatic or aromatic solvent, depending upon the end use of the composition.

A present composition also can contain a thickening or gelling agent. A thickening or gelling agent can be, for example, a polymer that is water soluble or that generates a colloidal solution in water. A thickening or gelling agent, therefore, can be, for example, polymers or copolymers unsaturated carboxylic acids or unsaturated esters, polysaccharide derivatives, gums, colloidal silicates, polyethylene glycols (PEG) and their derivatives, polyvinylpyrrolidones and their derivatives, polyacrylamides and their derivatives, polyacrylonitriles, hydrophilic silica gels, or mixtures thereof.

Specific thickening or gelling agents can be, for example, acrylic and/or methacrylic polymers or copolymers, vinylcarboxylic polymers, polyglyceryl acrylates or methacrylates, polyacrylamides derivatives, cellulose or starch derivatives, chitin derivatives, alginates, hyaluronic acid and its salts, chonodroitin sulphates, xanthan, gellan, Rhamsan, karaya or guar gum, carob flour, and colloidal aluminum magnesium silicates of the montmorillonite type.

Additional thickening or gelling agents include vinylcarboxylic polymers sold under the tradename CARBOPOL® (Goodrich), acrylic acid/ethyl acrylate copolymers, acrylic acid/stearyl methacrylate copolymers, carboxymethylcellulose, hydroxymethylcellulose, hydroxypropylcellulose, microcrystalline cellulose, hydroxypropyl guar, colloidal hectorites, bentonites, and the like.

The present compositions also can contain pigments, dyes, preservatives, hydrating agents, ultraviolet-absorbing agents, and the like.

The pigments can be inorganic pigments, organic pigments, or nacreous pigments. Inorganic pigments include, but are not limited to, titanium dioxide, black, yellow, red or brown iron oxide, manganese violet, ultramarine violet, ultramarine blue, chromium oxide, and the like. Among organic pigments, nonlimiting examples include D & C Red No. 3, No. 6, No. 7, No. 9, No. 13, No. 19, No. 21, No. 27, No. 30, or No. 36, or alternatively carbon black.

The nacreous pigments can be, for example, white nacreous pigments, such as mica coated with titanium oxide or with bismuth oxychloride. Colored nacreous pigments, such as titanium mica colored with iron oxides or with chromium oxide, titanium mica colored with an organic pigment of the above-mentioned type, or alternatively, nacreous pigments based on bismuth oxychloride, also can be used.

The dye can be, for example, a water-soluble dye, such as Ponceau disodium salt, alizarin green disodium salt, quinoline yellow, amaranth trisodium salt, tartazine disodium salt, rhodamine monosodium salt, fuchsin disodium salt, xanthophylls, and the like.

The present compositions also can contain fillers, especially clays of the montmorillonite, hectorite, or bentonite type, or other fillers, such as silicas, silicone powders, polyamides, or powdered polymethyl methacrylate. Various white fillers such as, for example, talc, kaolin, powdered TEFLON® (polytetrafluoroethylene), powdered polyethylene, powdered crosslinked poly-beta-alanine, and the like, also are useful.

Other classes of optional ingredients included in a present composition can be, but not limited to, pH adjusters, chelating agents, preservatives, buffering agents, foam stabilizers, opacifiers, and similar classes of ingredients known to persons skilled in the art.

Specific optional ingredients include inorganic phosphates, sulfates, and carbonates as buffering agents; EDTA and phosphates as chelating agents; and acids and bases as pH adjusters.

Nonlimiting examples of basic pH adjusters are ammonia; mono-, di-, and tri-alkyl amines; mono-, di-, and tri-alkanolamines; alkali metal and alkaline earth metal hydroxides; and mixtures thereof. Specific, nonlimiting examples of basic pH adjusters are ammonia; sodium, potassium, and lithium hydroxide; monoethanolamine; triethylamine; isopropanolamine; diethanolamine; and triethanolamine. Examples of acidic pH adjusters are the mineral acids and organic carboxylic acids. Nonlimiting examples of mineral acids are citric acid, hydrochloric acid, nitric acid, phosphoric acid, and sulfuric acid.

In the personal care area, a delivery system is incorporated into compositions designed as aftershave lotions, baby lotions, baby creams, baby shampoos, cosmetic basecoats and undercoats, bath capsules, bath oils, bath tablets, bath salts, bath soaps, blushers, colognes and toilet waters, cuticle softeners, depilatories, dusting and talcum powders, eye lotions, eye makeup products, eye makeup removers, eye shadows, eyebrow pencils, eyeliners, face powders, face, body, and hand creams and lotions, feminine hygiene deodorants, foot powders and sprays, cosmetic foundations, fragrance products, hormone creams and lotions, indoor tanning preparations, leg and body paints, lipsticks, makeup bases, makeup fixatives, makeup products, manicuring products, mascara, man's talcum, moisturizing creams and lotions, mouthwashes and breath fresheners, nail creams and lotions, nail extenders, nail polishes and enamels, night creams and lotions, antidandruff products, paste masks, perfumes, preshave lotions, noncoloring rinses, rouges, sachets, skin care products, skin fresheners, skin lighteners, suntan gels, suntan creams, suntan liquids, tonics, pharmaceutical skin treatment products, dressings, hair grooming aids, underarm deodorants, wave sets, and wrinkle smoothing creams and lotions.

In particular, a present delivery system can be incorporated into preparations, like lotions and after shaves; antiperspirants and astringents; bath preparations, like bath gelees, and bath oil; makeup preparations, like lipsticks, pomades, lip glosses, makeup foundations, face coloring sticks, nail hardeners, nail conditioners, eye covers, eye shadows, eye liners, mascaras, and cheek tints; shaving preparations, like moisturizers and emollients; skin care preparations, like hand lotions, vanishing creams, night creams, sunscreens, body lotions, facial creams, clay masks, moisturizing lotions, make-up removers, skin bleaching creams, antiacne preparations, antiaging preparations, and sebum control; analgesic and cortisonal steroid creams and preparations; insect repellants; anti-dandruff compositions; skin lightening compositions; facial masks and revitalizers; and self-tanning compositions.

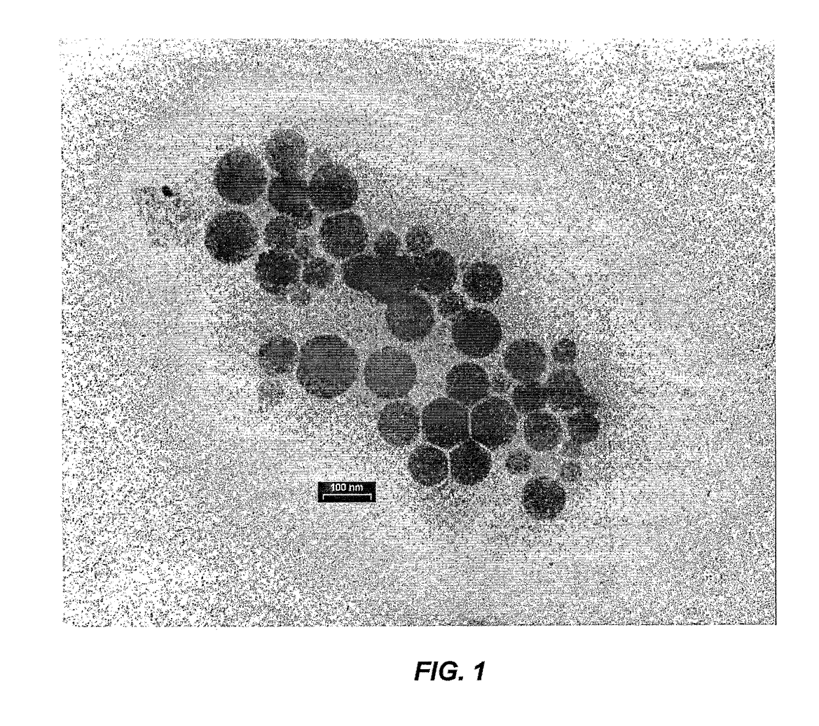

A delivery system of the present invention is illustrated in the Figures. FIG. 1 is a photograph of round vesicles present in the delivery system. In FIG. 1, an activated delivery system containing 10% oleic acid, 10% lecithin, 0.5% Vitamin E acetate, and 69.5% rice bran oil, by weight, was added to water containing a water-soluble black dye, i.e., phosphotungstic acid. One weight part of the delivery system was added to three weight parts of water. The resulting vesicles are permeable to the black dye, and the photograph of FIG. 1 shows round structural vesicles. The round, flexible vesicles of FIG. 1 allow penetration and the creation of reservoirs of the topically applied compound to the living epidermis and dermis.

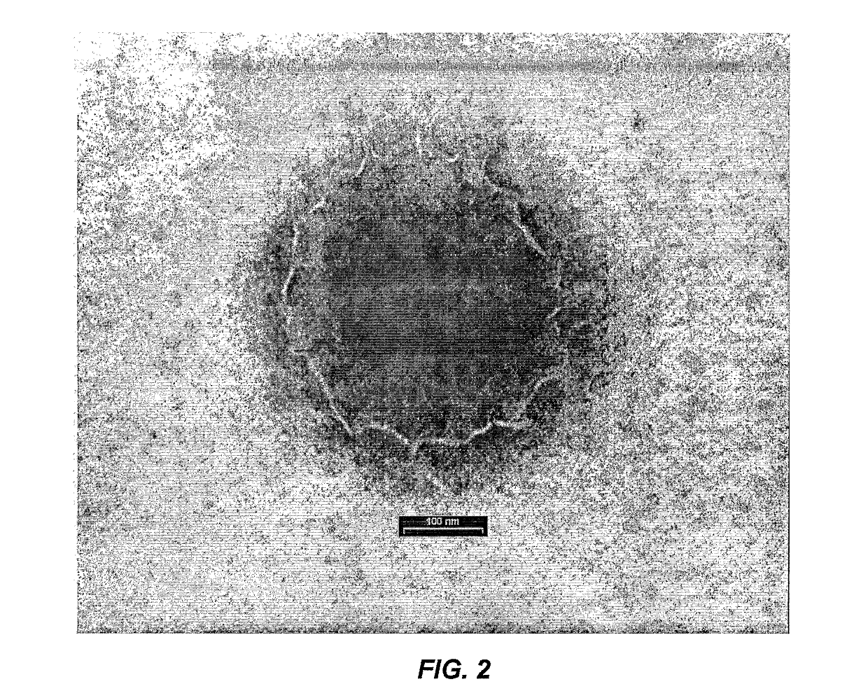

FIG. 2 illustrates partially ruptured vesicles of a present delivery system as described above, except for containing 20% oleic acid and 59.5% rice bran oil, by weight. These partially ruptured vesicles are capable of penetrating the surface of the skin into the epidermis and dermis. The partially ruptured vesicles, therefore, provide a controlled and sustained delivery of the topically applied compound into the epidermis or dermis, where the topically applied compound can perform its intended function for an extended time.

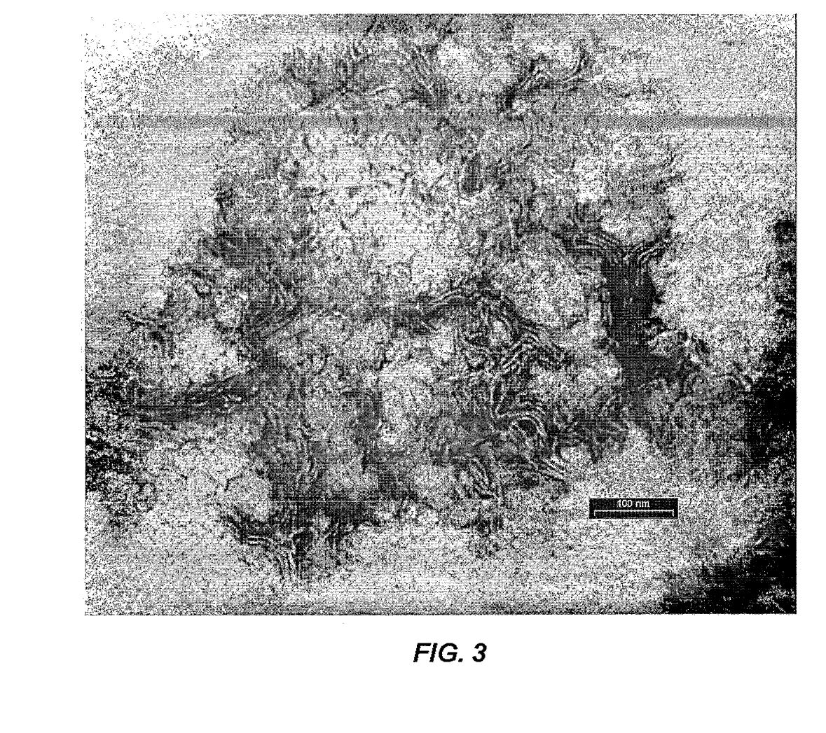

FIG. 3 illustrates completely ruptured vesicles of a present delivery system (as described above except for containing 30% oleic acid and 49.5% rice bran oil, by weight) forming lamellar sheets. These lamellar sheets are capable of retaining a reservoir of the topically applied compound in the stratum corneum.

Each of the round vesicles, partially ruptured vesicles, and lamellar sheets exist in a present activated delivery system. However, by a judicious selection of the fatty acid, phospholipid, and oil, and relative amounts of these compounds, the delivery system can be designed to increase the amount of one type of structure, and thereby control the site of delivery of the topically applied compound.

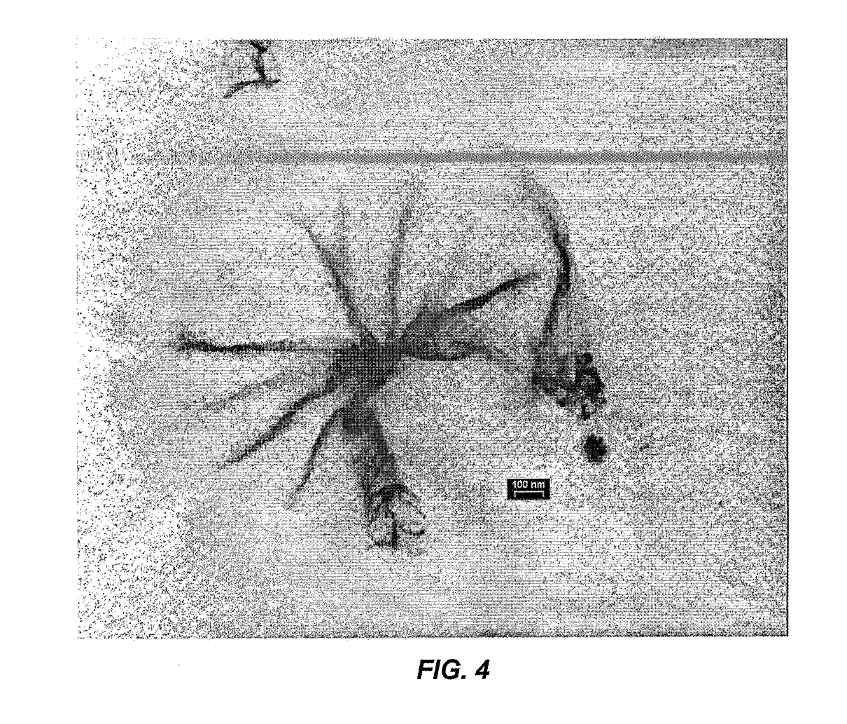

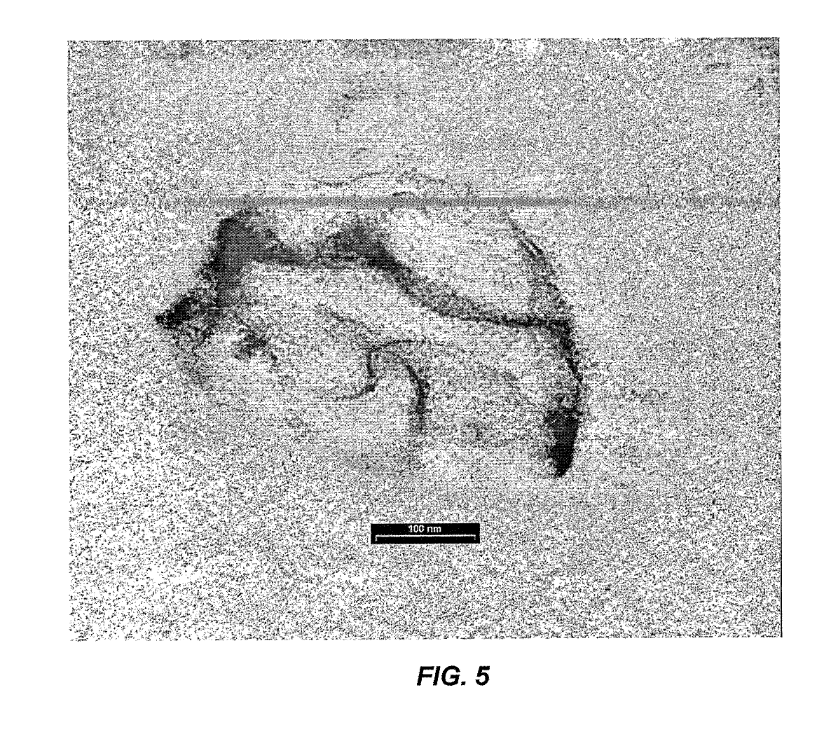

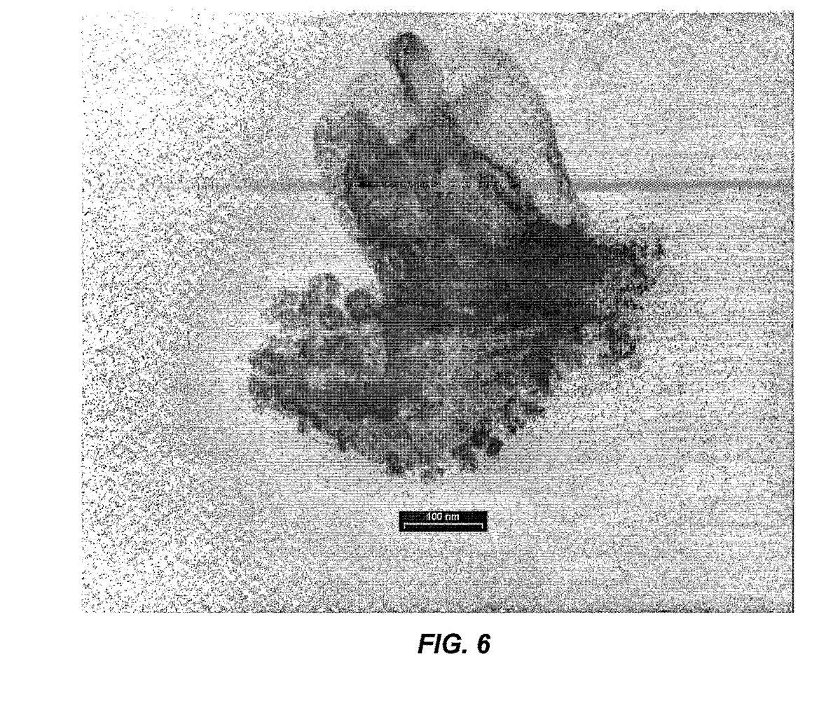

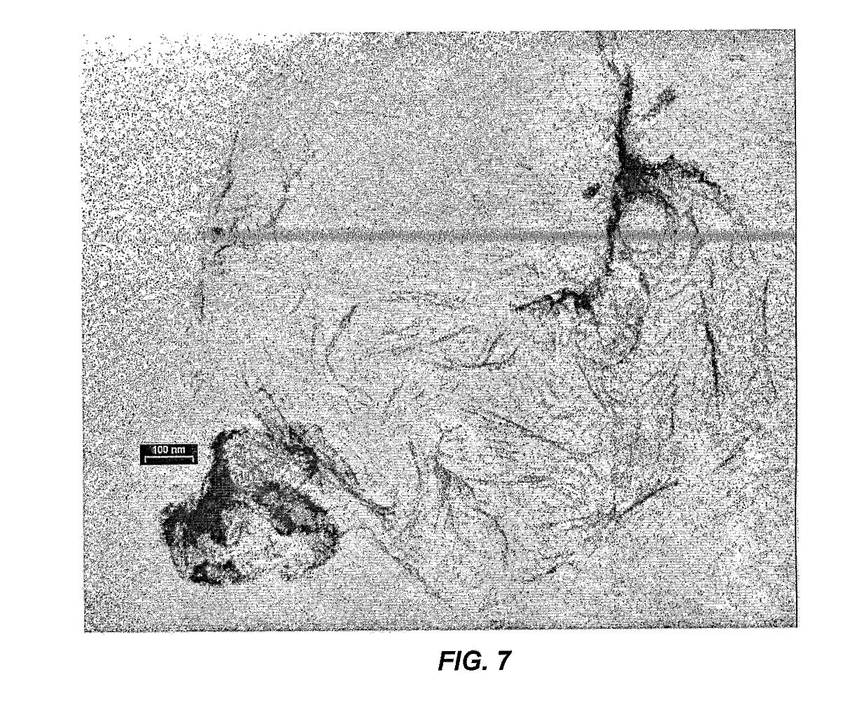

FIGS. 4 through 7 illustrate that, by increasing the amount of oleic acid in the delivery system, rupturing of the vesicles can be increased. In particular, the delivery system of FIG. 4 contains 10 wt % oleic acid; the delivery system of FIGS. 5 and 6 contain 20 wt % oleic acid; and the delivery system of FIG. 7 contains 30 wt % oleic acid. The delivery system of FIGS. 4-7 further contained 10 wt % lecithin and the balance rice bran oil. FIG. 7 shows the greatest degree of vesicle rupturing.

The following tests further illustrate the delivery system of the present invention.

### Sample Preparation

Samples were prepared by cold mixing lecithin (Z-3, ALC, Oxford, Conn.), oleic acid (Cognis, Cincinnati, Ohio), and jojoba oil (Lipo Chemicals, Paterson, N.J.). The oil phase then was mixed with water in different ratios. Activated delivery systems were prepared that individually contained 0% to 15% w/w oleic acid.

### Transmission Electron Microscopy (TEM)

TEM examines compositions by passing an electron beam through a test sample. The size, shape, and arrangement of the structures in the sample are observed using the TEM technique, as well as the relationships between the structures in the sample.

Samples were analyzed using TEM after a negative staining. In particular, a drop of diluted sample was mounted onto a copper grid, followed by applying a negative stain solution onto the sample, then drying for few minutes. The stains used in these tests were phosphotungstic acid (PTA) at 1% w/v concentration and uranyl acetate (UA). The individual samples then were observed using a Philips TEM CM 12 apparatus (EM, Eindhoven, Netherlands) at 110 kv-accelerated voltage.

### Differential Scanning Calorimetry (DSC)

DSC measures the energy needed to reduce the temperature difference between a test sample and an inert reference material to near zero. The procedure involves subjecting the sample and reference material to identical temperature regimes. The basic principle of DSC is that, in order to maintain the sample and reference material at the same temperature when the sample undergoes a physical transformation, such as a phase transition, more or less heat will be required. This amount of heat is measured and plotted versus a change in temperature. The DSC tests were performed using a TA instrumental thermal Analysis DSC (TA Instrumental, New Castle, Del.). In this test, samples were weighed accurately in an aluminum pan, then sealed tightly and mounted into a standard cell. Changes in heat flow versus changes in temperature were monitored over a heating/cooling cycle of −30° C. to 30° C.

### Skin Permeation

Radioactive oleic acid (MP Biomedicals) (2 μl) was added to 20 grams of each formulation, then admixed to achieve an uniform dispersion. Franz Diffusion cells (5.1 ml) were used to analyze the skin permeation profile for each formulation. A receptor compartment was filled with isotonic phosphate buffer containing 10% ethanol, and pieces of human cadaver skin were clamped between the donor and the receptor compartments and allowed to pre-hydrate for one hour. About 0.15 gm of each formulation was weighed and added to the donor compartment using a glass rod. Accurate weights of each formulation added to the donor compartments were recorded (for five replicate tests). The donor compartments and the sampling ports were covered tightly with PARAFILM™ and samples (300 μl) were withdrawn every hour for up to 8 hours. The receptor compartment solution was stirred continuously at 600 rpm and the entire unit was maintained at 37° C. After 8 hours, the skin was removed from the cells, then labeled and stored appropriately for tape stripping. Ethanol (3 ml) was used to collect the donor washings.

### Tape Stripping

Each piece of skin was weighed before tape stripping. Approximately one square inch pieces of Scotch Tape (810 Magic Tape™, 3M, St. Paul, Minn.) were cut and weighed. The tape then was pressed onto the skin, removed, and weighed again. Each piece of skin was tape-stripped in a similar manner seven times and the weights were recorded. Each tape stripped skin then was added to an empty scintillation vial. The remaining epidermis and dermis was weighed and added to scintillation vials. All pieces of dermis were digested in 2 ml of 0.3M sodium hydroxide for 12 hours at 80° C.

### Preparation of Samples for Reading

About 0.15 gm (accurate weights recorded) of the radioactive formulations were weighed and 10 ml of scintillation cocktail (ECOLITE™) was added to each. These counts were used to normalize the counts in the donor compartments. Scintillation cocktail (10 ml) was added to all receptor samples and the tape strips. Scintillation cocktail (5 ml) was added to the digested epidermis and dermis solutions. Scintillation cocktail (10 ml) was added to the donor washings. Appropriate standards and backgrounds were prepared and all samples were read for radioactive counts in a scintillation counter.

### Structural Properties

TEM photos showed the existence of three different structures, i.e., intact vesicles, ruptured vesicles (with nonuniform unilamellar membrane), and lamellar sheets. These three structures existed in all formulations. However, in each formutation, one structure dominated over the other two structures. See FIGS. 1-3, wherein the amount of oleic acid in the activated delivery systems was 5%, 10%, and 15%, by weight, respectively. A control formulation free of oleic acid had a TEM photo showing a structure typical of simple emulsion droplets.

### Thermodynamic Properties

Activated delivery systems were prepared containing oleic acid (0%, 5%, 10%, and 15%, by weight) and different phospholipids were used to adjust HLB values for stability. See Examples 3-5. The control formulation, which was free of oleic acid, was a simple emulsion. When measured by DSC, a present delivery system caused a decrease in Tₘ from 14.53° C. to 4.53° C. when the concentration of oleic acid was elevated from 0% to 5%, by weight. A further increase in oleic acid percentage to 10% and 15%, by weight, showed a slight increase in Tₘ to 5.16 and 6.25° C., respectively. Tₘ is a transition temperature wherein a phospholipid transforms from a gel state (ordered and rigid) to a liquid crystal-line state (random and fluid).

Crystallization temperature was a positive value (0.63° C.) for the control formulation (0% oleic acid) and a negative value (−1.52° C., −4.82° C., and −9.04° C.) for activated delivery systems containing oleic acid in amount of 5%, 10%, and 15%, by weight, respectively.

### Skin Penetration Behavior

When radiolabeled with oleic acid and detected in the skin, the four test samples demonstrated different skin penetration profiles. The test sample containing 15%, by weight, oleic acid had the highest deposition in the stratum corneum. The control formulation (0% oleic acid) showed less than half as much deposition in the stratum corneum.

A different behavior was observed when the target of interest was the live epidermis and the dermis. In this experiment, the 5%, by weight, oleic acid activated delivery system demonstrated the highest deposition, and the amount was doubled in comparison to the control formulation (0% oleic acid).

The amount of topically applied compound remaining on the skin shows that the delivery system allows for significant penetration into the skin. The advantage of a present activated delivery system is clear, e.g., the control formulation allowed only 38% penetration of the marker, whereas the present delivery systems containing 5%, 10%, 15%, by weight, oleic acid exhibited a substantially increased marker penetration of 61%, 56%, and 55%, respectively.

The above results demonstrate that a composition of the present invention results in significant structural and thermodynamic changes. Whereas a control formulation free of oleic acid showed typical oil-in-water (o/w) emulsion properties and a relatively high Tₘ. An activated delivery system of the present invention containing oleic acid reduced the Tₘ and generated “flexible” structures ranging from vesicles at 5%, by weight, oleic acid to lamellar sheets at 15%, by weight, oleic acid. These structural and thermodynamic changes further affected the interaction of a present delivery system with the skin. In particular, each delivery system exhibited a different dominant site for deposition of the radio-labeled marker. The lamellar sheets of the delivery system positioned the marker, or a topically applied compound, pre-dominantly in the upper layer of the skin, i.e., the stratum corneum, whereas the round, flexible vesicles allow for further penetration of the skin and deposited the marker, or a topically applied compound, predominantly in the live epidermis and dermis.

Obviously, many modifications and variations of the invention as hereinbefore set forth can be made without departing from the spirit and scope thereof and, therefore, only such limitations should be imposed as are indicated by the appended claims.

What is claimed is:

1. A delivery system for a topically applied compound comprising
(a) about 2% to about 50%, by weight, of a fatty acid;
(b) about 5% to about 50%, by weight, of a phospholipid;
(c) about 20% to about 90%, by weight, of an oil.

2. The delivery system of claim 1 wherein the system is free of a surfactant.

3. The delivery system of claim 1 wherein the fatty acid is present in an amount of about 5% to about 40%, by weight.

4. The delivery system of claim 1 wherein the fatty acid is present in an amount of about 10% to about 30%, by weight.

5. The delivery system of claim 1 wherein the fatty acid comprises a C₈ to C₂₆ fatty acid.

6. The delivery system of claim 1 wherein the fatty acid is selected from the group consisting of capric acid, caprylic acid, decanoic acid, lauric acid, behenic acid, tallow acid, caproic acid, myristic acid, oleic acid, linoleic acid, stearic acid, isostearic acid, tall oil acid, coconut acid, pelargonic acid, linolenic acid, ricinoleic acid, palmitic acid, hydroxystearic acid, linseed acid, undecylenic acid, soy acid, and mixtures thereof.

7. The delivery system of claim 1 wherein the phospholipid is present in an amount of about 5% to about 35%, by weight.

8. The delivery system of claim 1 wherein the phospholipid is present in an amount of about 10% to about 30%, by weight.

9. The delivery system of claim 1 wherein the phospholipid is selected from the group consisting of phosphatidylethanolamine, phosphatidylcholine, phosphatidylserine, phosphatidylinositol, phostidylglycerol, 3′-O-lysylphosphatidylglycerol, cardiolipin, sphingomyelin, and mixtures thereof.

10. The delivery system of claim 1 wherein the oil is present in an amount of about 30% to about 85%, by weight.

11. The delivery system of claim 1 wherein the oil is present in an amount of about 40% to about 80%, by weight.

12. The delivery system of claim 1 wherein the oil is selected from the group consisting of rice bran oil, lanolin oil, linseed oil, coconut oil, olive oil, menhaden oil, castor oil, soybean oil, tall oil, rapeseed oil, palm oil, neatsfoot oil, eucalyptus oil, peppermint oil, rose oil, clove oil, lemon oil, pine oil, orange oil, almond oil, apricot kernel oil, avocado oil, chaulmoogra oil, cherry pit oil, cocoa butter, cod liver oil, corn oil, cottonseed oil, egg oil, ethiodized oil, grape seed oil, hazel nut oil, hybrid safflower oil, hydrogenated castor oil, hydrogenated coconut oil, hydrogenated cottonseed oil, hydrogenated menhaden oil, hydrogenated palm kernel oil, hydrogenated palm oil, hydrogenated peanut oil, hydrogenated shark liver oil, hydrogenated soybean oil, hydrogenated vegetable oil, jojoba oil, mink oil, moringa oil, olive husk oil, palm kernel oil, palm oil, peach kernel oil, peanut oil, pengawar djambi oil, rice bran oil, safflower oil, sesame oil, shark liver oil, shea butter, sunflower seed oil, sweet almond oil, vegetable oil, walnut oil, wheat bran lipids, wheat germ oil, a hydrocarbon, mineral oil, 1-decene dimer, a polydecene, paraffin, petrolatum, an isoparaffin, dimethicone, dimethicone copolyol, dimethiconol, simethicone, phenyl trimethicone, stearoxy dimethicone, trimethylsilylamodimethicone, an alkyl dimethicone copolyol, a dimethicone, and mixtures thereof.

13. The delivery system of claim 1 wherein the weight ratio of fatty acid to phospholipid is about 0.2 to 1 to about 5 to 1.

14. The delivery system of claim 1 wherein the weight ratio of fatty acid to phospholipid is about 0.5 to 1 to about 2 to 1.

15. The delivery system of claim 1 wherein the weight ratio of fatty acid to phospholipid is about 0.8 to 1 to about 1.2 to 1.

16. The delivery system of claim 1 further comprising up to a total of 10%, by weight, of an optional ingredient selected from the group consisting of a surfactant, an antioxidant, a fragrance, a dye, a cholesterol ester, and mixtures thereof.

17. The delivery system of claim 1 wherein the fatty acid comprises oleic acid, linoleic acid, or mixtures thereof; the phospholipid comprises lecithin; and the oil comprises rice bran oil.

18. The delivery system of claim 1 wherein the system contains up to about 2%, by weight, of a surfactant.

19. An activated delivery system comprising a delivery system of claim 1 and water.

20. The activated delivery system of claim 19 wherein water comprises 50% to 99%, by weight, of the activated delivery system.

21. A composition for topical application to mammalian skin or hair comprising
(a) a delivery system of claim 1;
(b) a topically applied compound; and
(c) water.

22. The composition of claim 21 wherein the weight ratio of delivery system to water is about 1 to 1 to about 1 to 100.

23. The composition of claim 21 wherein the weight ratio of delivery system to water is about 1 to 1 to about 1 to 4.

24. The composition of claim 21 wherein the composition contains (a) round vesicles, (b) vesicles having a partially ruptured membrane, (c) vesicles having completely ruptured membrane creating lamellar sheets, or (d) mixtures containing at least two of (a), (b), and (c).

25. The composition of claim 21 wherein the topically applied compound is selected from the group consisting of a fragrance, a drug, a therapeutic agent, a deodorant, an antiperspirant compound, a skin conditioner, an antioxidant, an insect repellant, a counterirritant, a vitamin, a plant extract, a steroid, a skin-lightening compound, a self-tanning compound, an antibacterial compound, an antifungal compound, an antiinflammatory compound, a topical anesthetic, an epidermal lipid replacement a sunscreen, an optical brightener, a dermatitis or skin disease medication, and mixtures thereof.

26. The composition of claim 21 wherein the topically applied compound is selected from the group consisting of benzocaine, dyclonine hydrochloride, aloe vera, butamben picrate, lidocaine hydrochloride, xylocaine, providone-iodine, polymyxin b sulfate-bacitracin, zinc-neomycin sulfate-hydrocortisone, chloramphenicol, ethylbenzethonium chloride, erythromycin, lindane, benzoyl peroxide, erythromycin benzoyl peroxide, clindamycin phosphate, 5,7-dichloro-8-hydroxyquinoline, alclometasone dipropionate, betamethasone valerate, o-amino-p-toluenesulfonamide monoacetate, monobenzone, amcinonide, diflorasone diacetate, hydrocortisone, methylbenzethonium chloride, PEG-4 dilaurate, lanolin oil, petrolatum, mineral wax, butocouazole nitrate, haloprogin, clotrimazole, O-[(2-hydroxy-methyl)methyl]guanine, alclometasone dipropionate, betamethasone valerate, isopropyl myristate MSD, anthralin, methoxsalen, coal tar, 2-(acetyloxy)-9-fluoro-1′,2′,3′,4′-tetrahydro-1-hydroxypregna-1,4-dieno-[16,17-b]naphthalene-3,20-dione, 21-chloro-9-fluoro-1′,2′,3′,4′-tetrahydro-11b-hydroxypregna-1,4-dieno-[16z,17-b]naphthalene-3,20-dione, allantoin, salicylic acid, and mixtures thereof.

27. The composition of claim 21 wherein the active agent is selected from the group consisting of marigold extract, urea peroxide, thermus ferment, glycerin, an optical brightener, an anti-oxidant, a silicone, isopropyl myristate, ascorbic acid, retinol, salicylic acid, zinc pyrithione, benzophenone-3, a fragrance, glycolic acid, hyalauronic acid, hydrogen peroxide, a protein, an enzyme, tocopherol, butein, hydroquinone, kojic acid, jojoba oil, an alpha or beta hydroxy acid, and mixtures thereof.

28. The composition of claim 21 further comprising up to a total of 10%, by weight, of an optional ingredient selected from the group consisting of a surfactant, an antioxidant, a fragrance, a dye, a cholesterol ester, and mixtures thereof.

29. A method of treating mammalian skin comprising a step of contacting the skin with a composition comprising:
(a) a delivery system of claim 1;
(b) a topically applied compound; and
(c) water.

30. The method of claim 29 wherein the topically active compound penetrates a surface of the skin.

31. The method of claim 30 wherein the topically active compound is released in a sustained manner over time.

32. The method of claim 29 wherein the topically active compound penetrates a surface of the skin to an epidermis and dermis.

33. The method of claim 32 wherein the topically active compound is released in the epidermis and dermis in a sustained manner over time.
